(19) United States
(12) Patent Application Publication
(10) Pub. No.: US 2026/0294850 A1
(43) Pub. Date: Oct. 1, 2026

(54) OPHTHALMIC COOLING SOLUTIONS

(71) Applicant: Cayman Chemical Company Inc., Ann Arbor, MI (US)

(72) Inventors: Thushara KARUNASEKARA, Ann Arbor, MI (US); Shannon STACEY, Canton, MI (US); Mitchell DE LONG, Chapel Hill, NC (US)

(21) Appl. No.: 19/629,799

(22) Filed: Mar. 26, 2026

Related U.S. Application Data
(60) Provisional application No. 63/778,094, filed on Mar. 26, 2025.
(60) Provisional application No. 63/778,070, filed on Mar. 26, 2025.

(51) Int. Cl. A61K 31/167; A61K 9/00; A61K 9/08; A61K 31/045; A61K 45/00; A61K 47/02; A61K 47/18

(52) U.S. Cl. CPC A61K 31/167; A61K 9/0048; A61K 9/08; A61K 31/045; A61K 45/00; A61K 47/02; A61K 47/186

(57) ABSTRACT

The present disclosure provides an ophthalmic composition comprising one or more cooling agents; one or more of an Rx or OTC active ingredient; and about 0.01% weight/volume to about 10% weight/volume of a tonicity agent.

### CROSS REFERENCE TO RELATED APPLICATIONS

This application claims the benefit of priority under 35 U.S.C. § 119(e) of U.S. Provisional Application No. 63/778,094, filed Mar. 26, 2025, and U.S. Provisional Application No. 63/778,070, filed Mar. 26, 2025, the disclosures of which are incorporated by reference herein in their entireties.

### BACKGROUND

Ophthalmic compositions are needed which provide cooling independent of the active pharmaceutical ingredient, thus reducing discomfort upon administration. typically, ophthalmic agents are administered as solutions for both ease of dosing and reduced discomfort upon administration. however, not all active pharmaceutical ingredients are sufficiently tolerated as dosed. in particular, medicaments for easing the signs and symptoms of dry or irritated eyes might fail to bring immediate relief, or may in fact be irritating themselves. therefore, compositions that create an environment whereby the Rx or “over-the-counter” (OTC) active pharmaceutical ingredient(s) may be delivered and a cooling sensation created that minimizes discomfort upon administration is needed.

It has surprisingly been discovered that the major disadvantages of the use of a single cooling ingredient can be overcome by the combination, in specific ratios, of two or more cooling agents. The IUPAC name of WS-12 as its single most active diastereomer and enantiomer is: (1R,2S,5R)-N-(4-methoxyphenyl)-5-methyl-2-propan-2-ylcyclohexane-1-carboxamide.

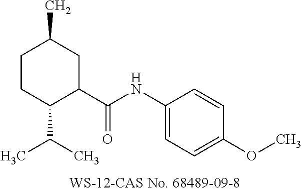 [SMILES: COc1ccc(NC(=O)C2C[C@H](C)CC[C@H]2C(C)C)cc1]

Without wishing to be bound by theory, it is thought that the specific ratios of WS-12 to menthol herein described are responsible for extending significantly the cooling sensation without the intense initial burst of cooling that is uncomfortable to many patients, and intolerable to some.

### SUMMARY

In one aspect, the present disclosure provides ophthalmic compositions for the treatment of an ophthalmic disease, condition or disorder, in a subject in need thereof, the composition comprising: menthol and WS-12; about 0.0001% weight/volume to about 1.0% weight/volume of an Rx or OTC API; and about 0.01% weight/volume to about 10% weight/volume of a tonicity agent.

In a second aspect, the present disclosure provides methods and kits for the treatment of an ophthalmic disease, condition or disorder, in a subject in need thereof, the kit comprising: one or more containers, the ophthalmic composition of the present disclosure, and instructions for use of the ophthalmic composition.

### DETAILED DESCRIPTION

In an aspect, the present disclosure provides an ophthalmic composition comprising one or more active Rx or OTC Active Pharmaceutical Ingredients (APIs) from about 0.001% weight/volume to about 5.0% weight/volume of a solution; and about 0.0001% weight/volume to about 1% weight/volume of one or more cooling agent(s). In another aspect, the present disclosure provides an ophthalmic composition comprising one or more active Rx or OTC APIs from about 0.001% weight/volume to about 5.0% weight/volume of a solution; and about 0.0001% weight/volume to about 1% weight/volume of two or more cooling agents. In another aspect, the present disclosure provides an ophthalmic composition comprising one or more active Rx or OTC APIs from about 0.001% weight/volume to about 5.0% weight/volume of a solution; and about 0.0001% weight/volume to about 1% weight/volume of the two cooling agents—a cooling agent of the menthol family and a cooling agent of WS family—in a ratio between 1:100 and 100:1 respectively, inclusive.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The conjunctive term “or” includes any and all combinations of one or more listed elements associated by the conjunctive term. For example, the phrase “an apparatus comprising A or B” may refer to an apparatus including A where B is not present, an apparatus including B where A is not present, or an apparatus where both A and B are present. The phrases “at least one of A, B, . . . and N” or “at least one of A, B, . . . N, or combinations thereof” are defined in the broadest sense to mean one or more elements selected from the group comprising A, B, . . . and N, that is to say, any combination of one or more of the elements A, B, . . . or N including any one element alone or in combination with one or more of the other elements which may also include, in combination, additional elements not listed.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### OTC APIs

For the purposes of the present disclosure, the phrase “OTC actives” and/or the phrase “OTC API's” refer to all of the compounds on the US FDA's monograph of allowed active ingredients in ophthalmic products. This list includes, but is not limited to, Zinc sulfate, Ephedrine hydrochloride, Naphazoline hydrochloride, Phenylephrine hydrochloride, Tetrahydrozoline hydrochloride.

### Compositions

In an aspect, the present disclosure provides an ophthalmic composition comprising WS-12 and menthol. WS-12 or menthol may be present in an amount of about 0.00001% w/v to about 0.06% w/v. Suitably, WS-12 or menthol may be present in an amount of about 0.001% w/v to about 0.003%. Importantly, the molar ratio of menthol to WS-12, when both are present, must be kept between 1:100 and 100:1, respectively. Other members of the WS family of cooling agents may be substituted for WS-12, such as WS-2, WS-3 and the like. Other members of the menthol family may be substituted for menthol such as borneol. For the purposes of this invention, the phrase “menthol” shall include (−) menthol, (+) menthol, racemic menthol, and borneol, in all proportions. The phrase “WS-12” shall include its enantiomers and diastereomers in all proportions, as well as the other members of the WS family of cooling agents, including but not limited to the longer lasting ones WS-5 and WS-10. WS-23 may also be used, particularly in combination with WS-12 and menthol when three cooling agents are used. The IUPAC name of WS-12 as its single most active diastereomer and enantiomer is: (1R,2S,5R)-N-(4-methoxyphenyl)-5-methyl-2-propan-2-ylcyclohexane-1-carboxamide. The IUPAC name for (−)-menthol, its single most active enantiomer and diastereomer is: (1R,2S,5R)-5-methyl-2-propan-2-ylcyclohexan-1-ol.

### Buffers

Suitable buffers include, but are not limited to, acetic acid, citric acid, carbonic acid, phosphoric acid, boric acid, the pharmaceutically acceptable salts thereof, and tromethamine. The buffer may be present in an amount of from about 0.01% weight/volume to about 1% weight/volume or about 0.1% w/v to about 0.9% w/v, or about 0.3% w/v to about 0.8% w/v, or about 0.4% w/v to about 0.6% w/v. Suitably, the buffer may be present in at least 0.01% w/v, at least 0.05% w/v, at least 0.1% w/v, at least 0.3% w/v, or at least 0.5% w/v. Suitably, the buffer may be present in an amount of no more than 1.0% w/v, no more than 0.8% w/v, no more than 0.6% w/v, or no more than 0.5% w/v.

### Tonicity Agents

The tonicity, or osmolality, of the product can be adjusted to hypotonicity, isotonicity, or hypertonicity relative to normal tears by use of conventional materials known in the art. Tonicity agents are typically ionic compounds. Examples of tonicity agents include, but are not limited to, sodium chloride, potassium chloride, mannitol, dextrose, glycerine, and propylene glycol. Suitably, the tonicity agent may be present in an amount of from about 0.01% weight/volume to about 10% weight/volume or about 1% w/v to about 10% w/v, or about 2.5% w/v to about 7.5% w/v, or about 4% w/v to about 6% w/v. Suitably, the tonicity agent may be present in at least 0.01% w/v, at least 0.05% w/v, at least 1% w/v, at least 2.5% w/v, at least 4% w/v, or at least 5% w/v. Suitably, the tonicity agent may be present in an amount of no more than 10% w/v, no more than 7.5% w/v, no more than 6% w/v, or no more than 5% w/v.

### Other Components

In embodiments, the composition may also contain pH adjusting agents in an amount sufficient to adjust the pH of the composition to between about 4.5 and about 7.5. Suitably, the composition has a pH of about 5.8 to about 7.2. Suitable pH adjusting agents include, but are not limited to, sodium hydroxide.

In embodiments, the composition may also contain an emulsifying agent. Suitable emulsifying agents include, but are not limited to, polyoxyl 40 stearate, polyethoxylated castor oil, and combinations thereof, and other agents known to one skilled in the art.

Ophthalmic products are typically packaged in unit dose or multidose form. Preservatives may be present to prevent or inhibit microbial contamination. Suitable preservatives include, but are not limited to, benzalkonium chloride, thimerosal, chlorobutanol, methyl paraben, propyl paraben, phenylethyl alcohol, edtate disodium, boric acid, sorbic acid, or other agents known to one skilled in the art. If present, the preservative may be present in an amount of about 0.001% w/v to about 1.0% w/v, or about 0.01% w/v to about 1.0% w/v, or about 0.1% w/v to about 1.0% w/v. Suitably, the preservative may be present in at least 0.001% w/v, at least 0.005% w/v at least 0.01% w/v, at least 0.05% w/v, at least 0.1% w/v, or at least 0.5% w/v. Suitably, the preservative may be present in an amount of no more than 1.0% w/v, no more than 0.9% w/v, no more than 0.5% w/v, or no more than 0.1% w/v.

In embodiments, racemic menthol may be used, or combinations of (−) menthol, (+) menthol and racemic menthol in all ratios. For the purposes of this invention all such combinations shall be considered “menthol”. In embodiments, borneol may also be used in place of menthol.

In embodiments, the composition may comprise a second active OTC pharmaceutical ingredient. The second active pharmaceutical ingredient may be chosen from the list in the well-known FDA monograph of OTC ophthalmic drugs (U.S. Food and Drug Administration Over-the-Counter (OTC) Monograph M018: Ophthalmic Drug Products for Over-the-Counter Human Use). If present, the second active OTC pharmaceutical ingredient may be present in an amount of about 0.001% w/v to about 1.0% w/v, or about 0.01% w/v to about 1.0% w/v, or about 0.1% w/v to about 1.0% w/v. Suitably, the second active OTC pharmaceutical ingredient may be present in at least 0.001% w/v, at least 0.005% w/v at least 0.01% w/v, at least 0.05% w/v, at least 0.1% w/v, or at least 0.5% w/v, at least 5% w/v. Suitably, the second active OTC pharmaceutical ingredient may be present in an amount of no more than 1.0% w/v, no more than 0.9% w/v, no more than 0.5% w/v, or no more than 0.1% w/v.

Other components commonly used in ophthalmic compositions, such as surfactants, comfort-enhancing agents, solubilizing agents, antioxidants, and stabilizing agents, may also be present.

### Formulations and Methods of Use

The ophthalmic formulations of the present disclosure may be formulated in various dosage forms suitable for topical ophthalmic delivery, including solutions, suspensions, emulsions, and gels. The compositions are suitably aqueous. The compositions may also be used, suitably modified, for otic use.

The present disclosure is also directed to methods of treating dry eye and other ophthalmic diseases or conditions. As used herein, “treat” or “treating” as used herein refers to administering a regimen to the subject, e.g., the administration of a compound or composition described herein, such that the condition or disorder or at least one symptom of the condition or disorder is healed, alleviated, relieved, altered, remedied, ameliorated, and/or improved. Treating includes administering an amount effective to alleviate, relieve, alter, remedy, ameliorate, improve and/or affect the disorder or the symptoms of the disorder. The treatment may inhibit deterioration or worsening of a symptom of a disorder.

The methods comprise topically applying to the affected eye(s) of the subject in need of treatment, for example, a human subject, a non-human mammal, (for example, a research animal used in ophthalmic disease research, (for example, a primate, a mouse, a rat, a ferret, a gerbil, a guinea pig, a rabbit, a goat, a pig), a domestic dog or a cat; a beneficial or therapeutically effective amount of a composition according to the present disclosure. The frequency and amount of dosage can be readily determined by one of skill in the art based on various clinical factors. The methods typically comprise topical application of one or two drops once or twice a day, but may be taken as often as 8 or more times a day.

### EXAMPLES

The present disclosure has multiple aspects, illustrated by the following non-limiting examples. In the various examples, the below materials and characterization techniques have been used.

### Example 1. Formulations with WS-12 and Menthol

Table 1 shows the composition of selected OTC formulations. A 500 mL volumetric flask containing 300 mL of water was heated to 45° C. on a hotplate. The required amounts of the OTC API, Benzalkonium Chloride, Boric Acid, Chlorobutanol, Edetate Disodium, Potassium Aspartate, and Sodium Chloride were quantitatively transferred into the volumetric flask. The solution was stirred at 300 rpm at 45° C. with a magnetic stir bar until complete dissolution.

### Preparing Cremophor RH40, WS-12, and Menthol Mixture:

Cremophor RH40 was heated to 35° C. to melt, and the required amount was weighed into a scintillation vial containing the prescribed amount of WS-12 and menthol. The vial was capped and placed on another hotplate at 50° C. until WS-12 and menthol dissolved completely. While maintaining the stirring, this mixture was quantitatively transferred into the 500 mL volumetric flask. The scintillation vial was rinsed twice with warm water (50° C.) and transferred to the 500 mL volumetric flask.

TABLE 1

| | Formulation A | Formulation B |
|---|---|---|
| Ingredient | % w/v | % w/v |
| WS-12 | 0.002% | 0.001% |
| Menthol | 0.008% | 0.005% |
| Phenylephrine hydrochloride | 0.025% | 0.03% |
| Povidone K90 | 2.0% | 2% |
| Cremophor RH40 | 1.9% | 1.5% |
| Benzalkonium Chloride | 0.01% | 0.01% |
| Boric Acid | 0.01% | 0.01% |
| Chlorobutanol | 0.02% | 0.02% |
| Edetate Disodium | 0.01% | 0.01% |
| Potassium Aspartate | 0.1% | 0.1% |
| Sodium Chloride | 0.75% | 0.75% |
| Purified Water | q.s. to 100% | 100% |
| Sodium Borate (5 g/dL) | q.s pH 7.0 | q.s pH 7.0 |

### Example 2. Formulations of Fixed-Dose Combination of Menthol and WS-12 and an OTC API

Table 2 shows the formulations of fixed-dose combination of menthol and WS-12. Formulations C & D were prepared in a labeled 150-milliliter (mL) plastic storage container. Approximately 100 milliliters (mL) of purified water were then added to bring the solution almost to 100% and the pH was adjusted to approximately 7.0.

TABLE 2

| | Formulation C | Formulation D |
|---|---|---|
| Ingredient | % w/w | % w/% w |
| WS-12 | 0.002% | 0.00105% |
| Menthol | 0.008% | 0.0045% |
| Naphazoline Hydrochloride | 0.025% | 0.03% |
| Povidone K90 | 2.0% | 2% |
| Cremophor RH40 | 1.9% | 1.5% |
| Benzalkonium Chloride | 0.01% | 0.01% |
| Boric Acid | 0.01% | 0.01% |
| Chlorobutanol | 0.02% | 0.02% |
| Edetate Disodium | 0.01% | 0.01% |
| Potassium Aspartate | 0.1% | 0.1% |
| Sodium Chloride | 0.75% | 0.75% |
| Purified Water | q.s. to 100% | 100% |
| Sodium Borate (5 g/dL) | q.s pH 7.0 | q.s pH 7.0 |

### Example 3. Preservative-Free, Fixed-Dose Combination of Menthol and WS-12 and an OTC API

TABLE 3

| | Formulation E | Formulation F |
|---|---|---|
| Ingredient | % w/w | % w/% w |
| WS-12 | 0.002% | 0.00105% |
| (−) Menthol | 0.008% | 0.0045% |
| Naphazoline Hydrochloride | 0.025% | 0.03% |
| Povidone K90 | 2.0% | 2% |
| Cremophor RH40 | 1.9% | 1.5% |
| Benzalkonium Chloride | 0.00% | 0.00% |
| Boric Acid | 0.01% | 0.01% |
| Chlorobutanol | 0.01% | 0.02% |
| Edetate Disodium | 0.01% | 0.01% |
| Potassium Aspartate | 0.1% | 0.1% |
| Sodium Chloride | 0.75% | 0.75% |
| Purified Water | q.s. to 100% | 100% |
| Sodium Borate (5 g/dL) | q.s pH 7.0 | q.s pH 7.0 |

When a drop of Formulation E is given to a person in need of a cooling sensation OTC product, the person experiences relief as well as the benefit of the active ingredient(s) in the formulation. When a drop of Formulation D is given to a human or a non-human animal in need of treatment, the human or non-human animal experiences the benefits of the solution.

It is understood that the foregoing detailed description and accompanying examples are merely illustrative and are not to be taken as limitations upon the scope of the invention, which is defined solely by the appended claims and their equivalents.

1. An ophthalmic composition, comprising:
menthol and WS-12;
about 0.0001% weight/volume to about 1.0% weight/volume of an Rx or OTC API; and
about 0.01% weight/volume to about 10% weight/volume of a tonicity agent.

2. The composition of claim 1, wherein the molar ratio of menthol to WS-12 is between 100:1 and 1:100 respectfully.

3. The composition of claim 1, wherein the menthol is predominantly (−) menthol.

4. The composition of claim 1, further comprising a preservative.

5. The composition of claim 4, wherein the preservative comprises benzalkonium chloride.

6. The composition of claim 1, wherein the patient is a human or a non-human mammal, including a domestic dog or cat.

7. The composition of claim 6, wherein the molar ratio of menthol to the at least one of WS-12, WS-5, WS-10 and WS-23 is between 100:1 and 1:100 respectively.

8. A method for treating an ophthalmic or otic condition or disorder in a subject in need thereof, the method comprising: administering a therapeutically effective amount of a composition comprising:
a) menthol and WS-12;
b) about 0.0001% weight/volume to about 1.0% weight/volume of an Rx or OTC API; and;
c) about 0.01% weight/volume to about 10% weight/volume of a tonicity agent.

9. The method of claim 8, wherein the molar ratio of menthol to WS-12 is between 100:1 and 1:100 respectively.

10. The method of claim 8, wherein the menthol is predominantly (−) menthol.

11. The method of claim 8, wherein the composition further comprising a preservative.

12. The method of claim 11, wherein the preservative comprises benzalkonium chloride.

13. The method of claim 8, wherein the subject is a human, a non-human mammal, a domestic dog or a cat.

14. A kit comprising, one or more containers, the ophthalmic composition of claim 1, and instructions for use of the ophthalmic composition.
